(19) United States
(12) Patent Application Publication
(10) Pub. No.: US 2011/0076338 A1
(43) Pub. Date: Mar. 31, 2011

(54) Composition for Skin Protection and Methods of Making Same

(76) Inventor: Tammy POWELL, Beaumont, TX (US)

(21) Appl. No.: 12/570,790

(22) Filed: Sep. 30, 2009

(51) Int. Cl. A61K 35/12; A61K 36/00

(52) U.S. Cl. 424/520; 424/725

(57) ABSTRACT

Skin care formulations having tamanu oil in amounts providing effective rates of protection for antimicrobial and anti-aging properties, including but not limited to promoting the formation of new tissue, scar healing and skin repair, anti-inflammatory, anti-bacterial, anti-fungal, anti-oxidant, and combinations thereof.

### BACKGROUND OF THE INVENTION

1. Field of the Invention

The present invention relates generally to compositions for skin protection and methods of formulation of same. Further, the present invention relates to natural components for compositions for skin protection applications, and methods relating thereto.

2. Description of the Prior Art

It is known in the health and beauty arts to provide skincare and skin protection, including for anti-aging effects resulting from the protective ingredients or formulations. However, many of the prior art skin protective formulations and compositions have artificial ingredients, or fail to provide effective protection without skin irritation or other issues that may exist with artificial ingredients. It is known to use tamanu oil in skincare products, more particularly as a conditioning or calmative agent used in hair and skin care products; however, the level and composition lacks multi-functional features and benefits of the present invention. Examples of relevant prior art include the following:

US Pub. No. 20070286838 describes a dermatological composition for treating skin, hair or nails containing tamanu oil. This publication provides for a composition that is 20-80% by weight of Tamanu Oil; 0.05-30% by weight of an unsaturated fatty acid; and 0.05-20% by weight of Arnica Oil.

U.S. Pat. No. 6,861,077 describes a process for protecting keratinous fiber or hair from damage using willowherb extract. Snail extract is listed in the patent specifications as a potential source of lectin that may be used in the practice of this invention. Also, multiple plant and animal extracts were included in the document.

Notably, none of the prior art patent documents describing a cosmetic composition containing both tamanu oil and snail extract, although separately both ingredients appear to be known agents in cosmetic compositions. Thus there remains a need for natural formulations of skincare products including tamanu oil and snail extract that are mild, safe and non-irritating for sensitive skin, while providing beneficial features of promoting the formation of new tissue, scar healing and skin repair, anti-inflammatory, anti-bacterial, anti-fungal, anti-oxidant qualities, etc.

### SUMMARY OF THE INVENTION

A first aspect of the present invention is to provide a product for skin protection and therapy including a composition having both Tamanu Oil and Snail Extract, and methods for formulating the same.

A second aspect of the present invention is to provide cosmetic products and formulations and methods therefore, including Tamanu oil and Snail extract for providing protection for promoting the formation of new tissue, scar healing and skin repair, anti-inflammatory, anti-bacterial, anti-fungal, anti-oxidant, and combinations thereof. Yet another aspect of the present invention is to provide these features and benefits in a natural product formulation(s) that are also mild, safe, and non-irritating to sensitive skin.

These and other aspects of the present invention will become apparent to those skilled in the art after a reading of the following description of the preferred embodiment when considered with the drawings, as they support the claimed invention.

### BRIEF DESCRIPTION OF THE DRAWINGS

The Figures provide tables of test results and features/benefits associated with the embodiment(s) of the present invention.

### DETAILED DESCRIPTION

The present invention is directed to a product for skin protection and therapy including Tamanu Oil and Snail Extract, and methods for formulating the same. More particularly, the present invention provides cosmetic products and formulations and methods therefore, including both Tamanu oil and Snail extract within the same composition for providing protection for promoting the formation of new tissue, scar healing and skin repair, anti-inflammatory, anti-bacterial, anti-fungal, anti-oxidant, and combinations thereof. Importantly, the present invention provides these features and benefits in natural product formulation(s) that are also mild, safe, and non-irritating to sensitive skin.

Referring now to the figures in general, these are provided for the purpose of describing a preferred embodiment of the invention and are not intended to limit the invention thereto.

The present invention provides for formulations of tamanu oil, also cleansers, and moisturizers. Formulations for products according to the present invention provide for skin care formulations having tamanu oil in amounts providing effective rates of protection for antimicrobial and anti-aging properties, including but not limited to promoting the formation of new tissue, scar healing and skin repair, anti-inflammatory, anti-bacterial, anti-fungal, anti-oxidant, and combinations thereof. Formulations according to the present invention include tamanu oil and snail extract, preferably in combination, wherein the tamanu oil is provided at dilutions of up to 1:1, 100,000 for efficacy against microbial contamination or cross-contamination.

Table 1 provides for Kill Rate Test Results for various compositions including Tamanu oil against E. coli at 6 hours and 24 hours, and against S. aureus at 6 hours and 24 hours; these compositions included Tamanu oil with Ethoxydiglycol, Tamanu Tattoo Care Cream, and Tamanu oil Pure Virgin, wherein for every case there was an effective result providing for “total kill” at the 24 h test sample point. Thus, Table 1 provides data supporting the efficacy of Tamanu oil for antibacterial properties in the compositions and formulations for skin care products according to the present invention. Table 2 provides data from testing showing the Effect of Tamanu Oil for Anti-bacterial properties for a broader variety of strains and types of bacterial and microorganisms than illustrated by the test results shown in Table 1.

Preferably, the tamanu oil is provided in an effective concentration, more preferably in a minimum inhibitory concentration (MIC) for the final product for skin care applications, as tested by a microdilution method. Test results for the present invention efficacy included each bacterium tested in triplicate, wherein a stock solution for each product of 0.1 g/ml in phosphate buffered saline (PBS) with dilutions at 10⁻² (10 mg/ml), 10⁻³ (1 mg/ml), 10⁻⁴ (0.10 mg/ml), 10⁻⁵ (0.010 mg/ml), and 10⁻⁶ (1 μg/ml), wherein each strain was grown to stationary phase and inoculated at a final concentration of 10⁵ into the tamanu product dilution, incubated at 37° C. with shaking. The MICs for “Turf Grass”, “Locker Room”, and “Clothing Spray” were tested by a microdilution method using a percent by volume in PBS, with each bacterium tested in duplicate; strains were grown to stationary phase an inoculated at a 10⁵ concentration into the tamanu product dilution, incubated at 37° C. with shaking. “Turf Grass” was tested effective against Stphylococcus aureus and S. aureus MRSA at concentrations as low as 5% by volume tamanu oil. “Locker Room” is effective against both strains at concentrations higher than about 50% by volume.

Table 2 illustrates test results for product formulations according to the present invention, including the “Turf Grass Disinfectant” and “Locker Room Disinfectant”. In the Table 2, (-) indicates no growth (i.e., TIA or tamanu oil dilution product being extremely effective at this dilution); P indicates few scattered colonies (i.e., TIA highly effective at this dilution); G indicates numerous or confluent colonies (i.e., TIA highly effective at this dilution).

Table 3 illustrates “Summary results of the efficacy of The Invisible Armor antibacterial lotion on the following microorganisms:” wherein the tamanu oil dilution is included in a lotion product composition.

Table 4 illustrates “Invisible Armor Report: Invisible armor with 1.5% EDTA” showing reading the results: −2=1:100 dilution of TIA; −6=1:1,000,000 dilution of TIA.

Table 5 illustrates “Summary” for “TIA (1.5% EDTA)”.

Table 6 illustrates “Summary” for “TIA (natural antibacterial agent)”.

Regarding properties for skin smoothness, the compositions and skin care products according to the present invention provide for improved skin smoothness based upon assessments using PRIMOS, which provides a phase-shifting rapid in vivo measurement of the skin using a non-contact measurement device providing real-time 3D measurement of the microtopography of human skin. Results were based on active image triangulation to assess skin smoothness based on its topography. Testing included placebo plus four (4) concentrations of the compositions formulated according to the present invention, wherein the test products were applied twice daily over a period of 28 days, with 20 subjects having an average age of 44.8 years.

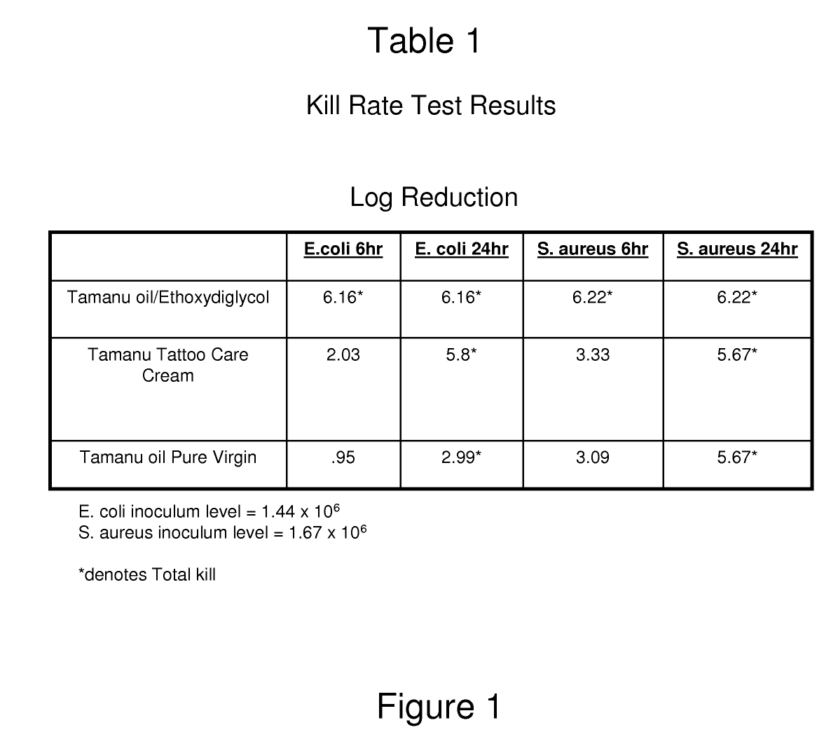

FIG. 1 is a bar chart illustrating “Increase in Skin Smoothness relative to initial conditions and to untreated” illustrating the increase in skin smoothness with increased tamanu oil concentration up to a point, showing optimized efficacy around 5% tamanu oil after 14 days and after 28 days still having optimized efficacy around 5% tamanu oil, comparable to 100% tamanu oil at around 14.0% increase in skin smoothness.

Regarding safety, testing for 100% pure tamanu oil on 50 human subjects via a repeat-insult test patch of skin indicated that all subjects (100%) showed no evidence of any effect; this safety testing was conducted by AMA Laboratories, Inc.

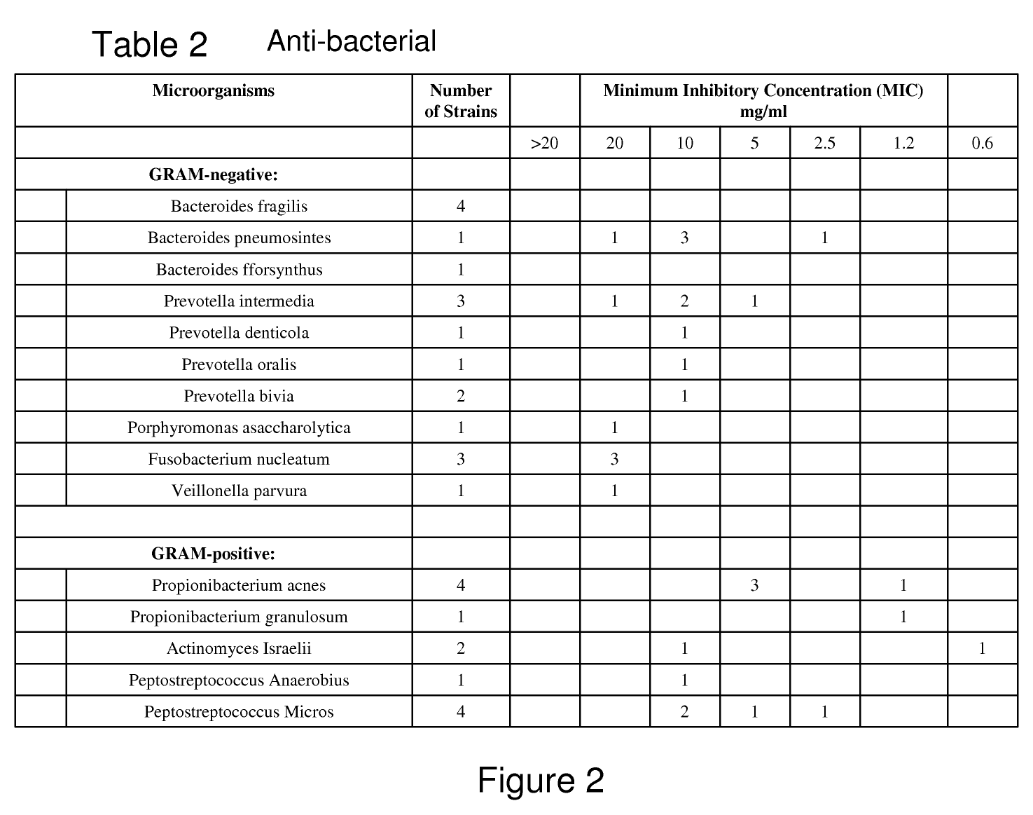

FIG. 2 is a bar chart illustrating “Increase in Skin Elasticity relative to initial conditions and to untreated” illustrating the increase in skin smoothness with increased tamanu oil concentration up to a point, showing optimized efficacy between about 2.5% and about 5% tamanu oil after 14 days, and after 28 days still having optimized efficacy around 2.5% tamanu oil, better than about 100% tamanu oil; these data illustrate optimized efficacy at 14 days at around over 6.0% increase in skin elasticity and almost about 14.0% increase in skin elasticity.

Another beneficial property of the compositions formulated according to the present invention includes scar healing. Testing for 100% pure tamanu oil on six (6) human subjects having obviously aged scars (from wounds inflicted and healed leaving a scar for at least one year or more) via qualitative visual ratings of scar appearance, including color, roughness, difference from normal surrounding skin, and via quantitative measurements including scar length and width size measurements relative to baseline values, wherein the product was applied twice daily over a period of nine (9) weeks. FIG. 3 is a line graph showing the “Effect of Tamanu Oil on Aged Scars” for 100% tamanu oil, illustrating continued improvement over 9 weeks of evaluation, considering the mean visual score of the scarred skin, beginning around 3.5 and decreasing to about 1.5 over that timeline.

Yet another beneficial property of the compositions formulated according to the present invention includes skin hydration. Testing for quantitative results for hydration state of the stratum corneum assessed by measuring the electrical capacitance of the skin surface, as measured by five (5) different measurements using a Corneometer CM 825 PC for applications of a placebo plus four (4) concentrations of tamanu oil levels in compositions according to the present invention, wherein the test products were applied twice daily over a period of 28 days, with 20 subjects having an average age of 44.8 years.

FIG. 4 is a bar chart illustrating “Decrease in Wrinkle Depth relative to initial conditions” illustrating the decrease in wrinkle depth with tamanu oil concentration, showing optimized efficacy at about 5% tamanu oil after 14 days and after 28 days still having optimized efficacy at about 5% tamanu oil, comparable to 100% tamanu oil at around 6.0% increase in skin smoothness at 14 days and better than 100% tamanu oil at around 14% at 28 days.

Regarding the formulations of the present invention, preferably the tamanu oil is provided at optimized concentrations for the formulation and application, based upon the foregoing test results. According to the present invention, lotions, gels, creams, and other liquid-based formulations include between about 0.5% to about 10% tamanu oil, more preferably about 5% tamanu oil by weight in the formulation for optimized results in skin care applications.

Certain modifications and improvements will occur to those skilled in the art upon a reading of the foregoing description. By way of example but not limitation, an alternative embodiment with triclosan may be considered for combination with the snail oil instead of tamanu oil. Preferably, triclosan is provided at about 0.5% by weight in the formulation alternative to tamanu oil. In addition to skin care, preferably the formulations of the present invention are adapted for hair and cosmetics applications as well. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the present invention.

What is claimed is:

1. A skin care formulation for treatment of skin conditions comprising tamanu oil in amounts for providing effective rates of protection for antimicrobial and anti-aging properties.

2. The formulation of claim 1, wherein the tamanu oil is provided in concentrations between about 0.5% and about 10%.

3. The formulation of claim 1, wherein the tamanu oil is provided at a concentration of about 5%, thereby providing optimized efficacy for skin treatment.

4. The formulation of claim 1, wherein the formulation actively promotes the formation of new tissue, scar healing and skin repair, anti-inflammatory, anti-bacterial, anti-fungal, anti-oxidant, and combinations thereof.

5. The formulation of claim 1, further including snail oil.

6. A method for treating skin conditions comprising the steps of:
providing a skin care formulation having tamanu oil in concentrations effective for protecting against microbial infections and aging; and
applying the formulation to a portion of skin for a period of at least one day.

7. The method for treating skin conditions of claim 6, further including repeating the process for up to at least 14 days.

8. The method of claim 6, further including the steps of repeating the process for up to 28 days.
